(19) United States
(12) Patent Application Publication
(10) Pub. No.: US 2008/0147018 A1
(43) Pub. Date: Jun. 19, 2008

(54) LAPAROSCOPIC CANNULA WITH CAMERA AND LIGHTING

(76) Inventors: John R. Squilla, Rochester, NY (US); Joseph P. Di Vincenzo, Rochester, NY (US); Nelson A. Blish, Rochester, NY (US)

Correspondence Address: Carestream Health Inc,, 150 Verona Street, Rochester, NY, 14608, US

(21) Appl. No.: 11/611,405

(22) Filed: Dec. 15, 2006

(51) Int. Cl. A61M 25/00

(52) U.S. Cl. 604/264

(57) ABSTRACT

A medical cannula (100) comprises a first sleeve (800) and a center punch which fits within the sleeve for providing an opening in a body for the sleeve and the center punch. At the least one camera channel (120) is in the sleeve for viewing inside the body.

### FIELD OF THE INVENTION

This invention relates in general to medical devices, and in particular to a cannula for use in laparoscopic surgery.

### BACKGROUND OF THE INVENTION

Laparoscopic surgery is becoming increasingly used to perform surgical procedures that have traditionally been performed using open surgical procedures. The patient benefits of having a laparoscopic procedure (instead of an open surgery procedure) are well known and include reduced trauma to patient tissue, smaller scars, less post-surgical pain, and faster recovery and return to regular activity levels.

The benefits to surgeons performing laparoscopic procedures include improved outcomes and greater patient satisfaction. However, laparoscopic procedures require the surgeon to access the surgical site inside the patient through a series of small (usually 12 mm diameter or less) incisions, significantly limiting access and visibility of the surgical site. In addition, the internal patient body cavity around the surgical site is generally insufflated with carbon dioxide to create an air space in which the surgeon can view the surgical site and move their laparoscopic surgical instruments inside the expanded body cavity.

To form an airtight seal to retain the insufflated gas while allowing insertion and removal of laparoscopic surgical instruments, a trocar is used to create an opening in the body cavity and a surgical cannula is inserted into each small incision. Currently used surgical cannulas consist of a tube through which surgical instruments can be inserted, a sharp piercing component (i.e. the trocar) that is inserted into the cannula tube to pierce the patient's skin and muscle and a port through which the surgical instruments can be inserted into the body. Surgical instruments may then be inserted and extracted through each cannula. The number of incisions is determined by the numbers surgical instruments to be used simultaneously and the surgical paths needed to access the surgical site.

A cannula generally has a port for connecting a gas source to insufflate the patient and a switch enabling the gas to be exhausted to reduce the volume of gas inside the patient body cavity at the conclusion of surgery. The cannula is designed to provide an airtight seal between the cannula and the tissue around the incision as well as between the cannula and the surgical instrument inserted through the cannula port into the body cavity.

During use, the cannula maintains the airtight seal while acting as a fulcrum for the surgical instruments inserted through the cannula. These surgical instruments are well known in the art and typically include surgical instruments for cutting, cauterizing, grabbing and blunt dissection, ablating, irrigation and suction, suturing, diseased tissue extraction and various laparoscopes and light sources to enable the surgeon to observe the surgical site inside the body cavity. Since the surgical instruments are inserted through the cannula and use the cannula as a fulcrum, the distal end of the cannula (i.e. the end inside the patient body cavity) is always pointed in the same direction as the surgical tool inserted through that cannula.

Laparoscopes traditionally are designed like a small telescope through which the surgeon can directly view the surgical site or with a video camera attachment for viewing the surgical site. The video from a laparoscope camera is usually displayed on a TV or computer monitor. The surgeon observes the surgical site indirectly using the TV or computer monitor.

Traditionally, both the laparoscope camera and the light source are connected to the laparoscope external to the body cavity (e.g. Karl Storz example). This is due largely to the physical size of the camera and light source as well as power consumption requirements (i.e. creating heat generation and safety issues) that required these components to be outside the patient body cavity.

It is customary for laparoscopes to provide a port to attach an external light source (e.g. a Xenon light source) using a fiber optic light cable.

With the increasing miniaturization and reduced energy consumption of modern CCD image sensors, it has become possible to design laparoscopes with the video camera sensor on the distal end of the laparoscope (i.e. located inside the patient body cavity). For example, Olympus introduced their Endo-Eye laparoscope.

At least four (4) surgical ports are usually required for: the laparoscope, irrigation/suction, left hand surgical tool and right hand surgical tool. Usually the primary surgeon manipulates the left hand and right hand surgical tool while the assistant surgeon positions the laparoscope and uses the irrigation/suction device as needed. Laparoscope holders and other equipment have been developed to enable the surgeons to hold surgical instruments stationary, freeing their hands for other tasks. Communication and coordination is therefore required between the primary surgeon and the assistant surgeon to ensure that the laparoscope, operated by the assistant surgeon, is illuminating and viewing the area that the main surgeon needs to see to operate the left hand and right hand surgical tools.

Fewer surgical ports can be used if required by alternating surgical instruments inserted through that single port (e.g. withdrawing the left hand surgical instrument and temporarily using the port for the irrigation/suction device). More surgical ports may be needed to accommodate additional surgical tools or surgical paths.

As new laparoscopic surgical equipment is developed, new methods to introduce these devices into the body cavity will be desirable without requiring additional patient incisions. For example, optical molecular imaging technology can be used to use the difference in autofluorescent properties between health and diseased tissue to help the surgeon define the surgical margin to ensure complete diseased tissue removal. This generally requires the use of an additional autofluorescence illumination source with specified frequencies and narrowband filtering of the resulting video of the diseased tissue illuminated by the autofluorescence illumination source.

It is desirable to accommodate additional surgical instruments capable of illuminating and imaging the surgical site without increasing the number of surgical ports. Additionally, a mechanism that easily enables surgeons to ensure the illumination and video sensing are aimed at the area of interest in the surgical site will reduce the workflow complexity caused by the introduction of new technology-enabled surgical instruments.

LED light sources and light channels to deliver the illumination are well known in the art.

U.S. Pat. No. 6,387,044 (Tachibana et al.) describes a surgical cannula with a light guide for illuminating the object to be observed while also providing a tubular member (cylindrical container) into which an endoscope can be inserted. Control of the light guide angle at the distal end of the cannula and the use of diffusers and prisms at the distal end of the light guide are described as a means to reduce light source halation. Further described is a mechanism to insert the endoscope into the cannula and temporarily join these components together using an association claw engaged into corresponding association grooves. The key benefits of the separation of the cannula and endoscope are described as allowing more flexibility in wide-angle illumination control as well as independent means of sterilizing the light guides and the endoscope.

There are 1 mm cameras in the art as demonstrated by M. Last et al., “Towards a 1 mm³ Camera—The Field Stitching Micromirror” Berkeley Sensor and Actuator Center, University of California, Berkeley. This, combined with the art of micro electromechanical systems (MEMS), has made the insertion of complex camera and other computer systems common in the art.

### SUMMARY OF THE INVENTION

Briefly, according to one aspect of the present invention describes a device and method for a new surgical cannula that is capable of providing surgical site illumination and image capture, in addition to traditional surgical cannula functionality. The invention has the benefit of enabling surgical ports used for surgical instruments to also provide this additional illumination and image capture capability without the need to increase the number of surgical ports, as would be required if the illumination and image capture were a separate standalone surgical instrument. Further, the surgical cannula geometry, with respect to the surgical instrument inserted through it, coupled with the fulcrum provided by the cannula has the added benefit of always pointing the cannula illumination source(s) and image capture sensor(s) in the same direction as its corresponding surgical instrument. This is accomplished by the surgeons simply moving the surgical instrument inserted through the cannula without the need to explicitly coordinate the field of view of an independent laparoscope with the surgical instrument.

The invention and its objects and advantages will become more apparent in the detailed description of the preferred embodiment presented below.

### BRIEF DESCRIPTION OF THE DRAWINGS

FIGS. 1a-1c are an embodiment of a cannula with cameras and lighting embedded within the walls.

FIG. 2 is an embodiment where the lighting is a ring light encompassing the entire front wall of the cannula.

FIG. 3 is a stereo camera configuration within a single cannula.

FIG. 4a shows top view of lighting channels and camera conduits.

FIG. 4b shows a side view of lighting channels and camera conduits.

FIG. 5a is a cross-sectional view of an image capture sensor with a telescope ring.

FIG. 5b shows a telescoping sleeve in profile.

FIGS. 6a and 6b shows a rear view showing camera and lighting connectors.

FIG. 7 is a diagram of positioning system within cannula wall.

FIG. 8 shows an inner sleeve that rotates within the cannula.

### DETAILED DESCRIPTION OF THE INVENTION

The present invention will be directed in particular to elements forming part of, or in cooperation more directly with the apparatus in accordance with the present invention. It is to be understood that elements not specifically shown or described may take various forms well known to those skilled in the art.

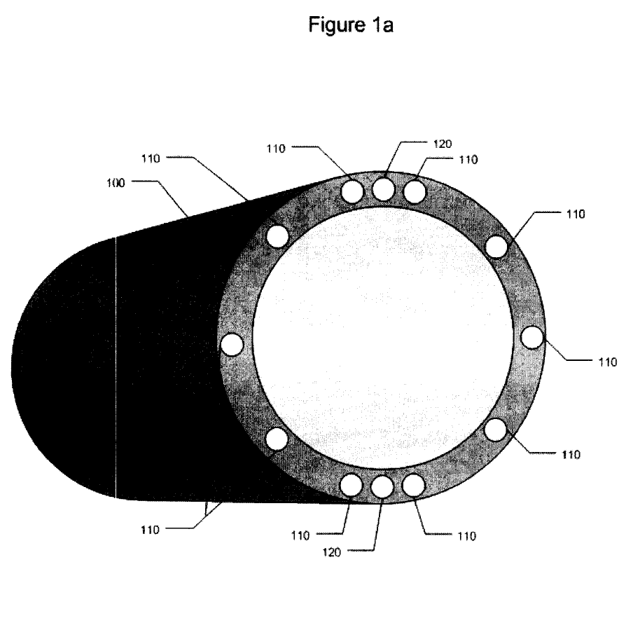

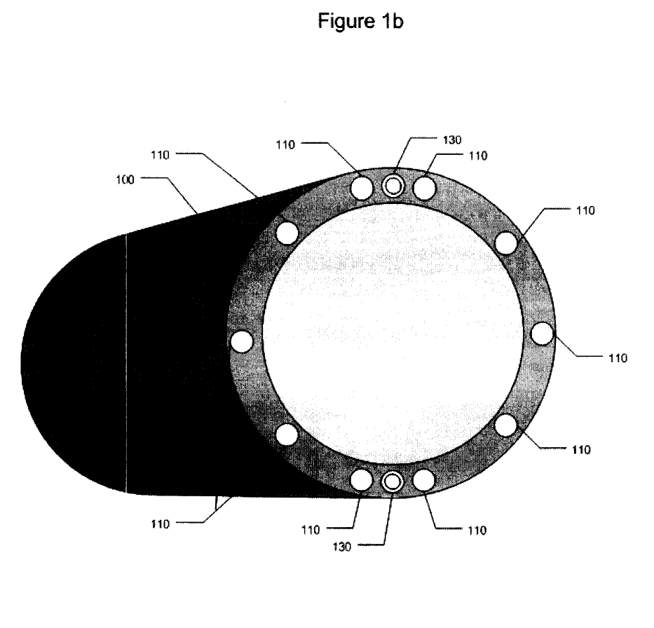

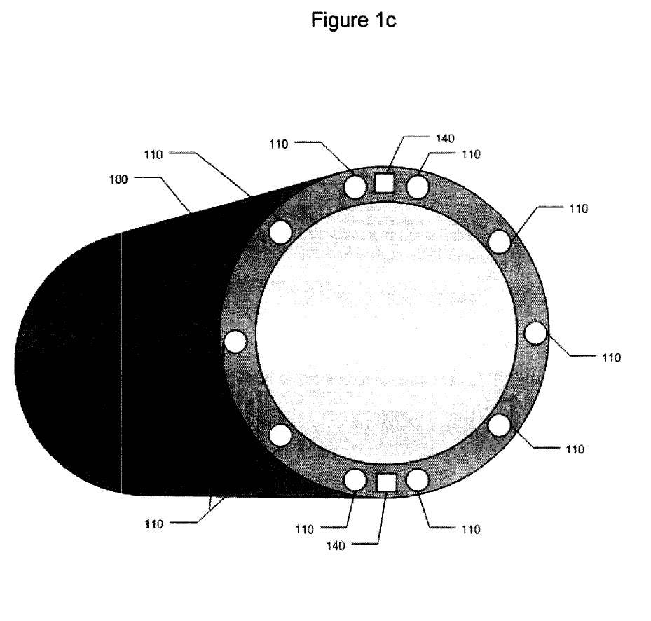

The most basic implementation of this invention is shown in FIG. 1. The function of a traditional surgical cannula has been extended to include the ability to provide illumination into the body cavity by means of the cannula body. The illumination geometry will depend on the desired optical properties of the illumination.

FIG. 1a shows the cannula's 100 distal end with discrete light bundles 110. Discrete light bundles have properties similar to point light sources, illuminating a small area with fall-off in illumination toward the edges of the illuminated area. A camera 120 is installed within the wall of the cannula 100. Two cameras 120 channels are shown to allow for the camera connection to be changed as the need for a different view presents itself or provides for the need for multiple views of the target area. This does away with the need for a special cannula for the camera and also allows for a direct view option from the perception of the tools used. Optionally, a single light or a single camera can be used as an alternative embodiment. The present invention also allows for more than two cameras as needed.

FIG. 1 a also shows the distal end of the cannula with use of multiple camera channels 120 of the present invention. The number and type of image capture sensors are determined by the optical geometry required by the viewing system. For example, two camera channels 120 can be placed 180 degrees apart around the circumference of the cannula (i.e. located at opposite ends of the geometric chord of the circumference of a circular cannula corresponding to the diameter of the cannula). This geometry would be desirable where maximal field of view would be provided by the joint outputs of both sensors.

FIG. 1b shows a camera 130 itself within the cannula. Miniaturization and cost improvements may allow this embodiment to be preferred for minimizing connections and components.

FIG. 1c shows an image sensor as a component to the camera to separate the sensor from the rest of the camera to allow more flexibility (articulation) and the prevention of light loss.

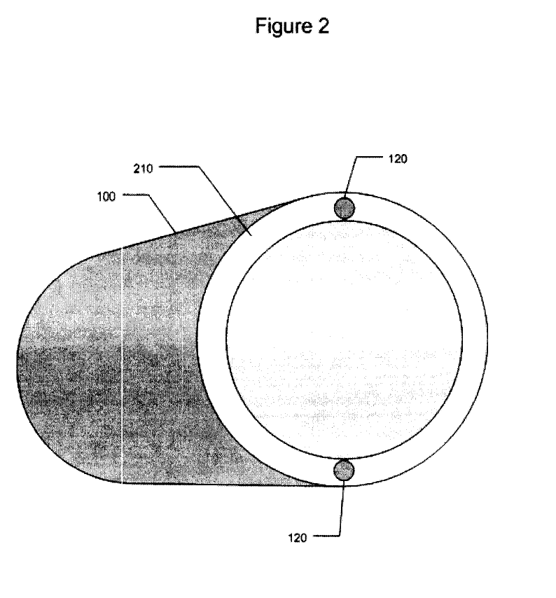

FIG. 2 shows the distal end of the surgical cannula 100 that is inserted into the patient's body (i.e. the cannula's distal end) with a ring light 210 geometry that would provide even illumination levels around the circumference of the cannula through which the surgical instrument is inserted. This optical geometry is well known and provides a relatively uniform illumination source. Alternatively, a half ring light can be used to provide illumination in the area of one of the cameras 120.

With the addition of optics with the cannula illumination geometry, it is possible to further define the illumination optical properties. For example, optics can be used to focus the illumination into a tight beam to maximize illumination brightness while illuminating a small area. Alternately, optics could be used to diffuse the illumination. Optics can also be used to control the direction of illumination. For example, the optics can focus the beam straight ahead or at an angle depending on the optical geometry required between the illumination source and the image sensor.

In another embodiment, on-board illumination source of the present invention provided by the surgical cannula. In this case, no external connection to an external illumination source would be required. Instead, a connection to a power source required by the on-cannula illumination source is shown. On-board illumination options are becoming increasingly practical due to the significant illumination source miniaturization and power consumption reduction supported recent technology advances (e.g. white light emitting diodes (LEDs) and semiconductor laser diodes).

Alternatively, a single image capture sensor 140 can be placed in the cannula's distal end. A cannula with single image capture sensor can be used without requiring cannula supplied illumination as previously described and covered by the present invention. For example, sufficient illumination may be provided by alternative illumination sources (e.g. the illumination source of a traditional laparoscope) facilitating the use of a reduced cost cannula of the present invention's design that does not have the ability to provide cannula-based illumination.

The surgical instrument located between these two image capture sensors would tend to prevent the individual image capture sensor fields of view from overlapping. This is analogous to living biological systems (e.g. wild rabbits) that have significantly wide field of vision with eyes on opposite sides of their heads. As is known from biology, this benefit of maximal field of vision, highly valued by natural selection to avoid predators, comes with the tradeoff that these animals lack stereovision capability.

It is well known in the art that stereovision requires an adequate amount of overlap between two camera's fields of view. This overlap is require to establish correspondence between objects that are located in both captured images but at different positions in each camera's field of view. This difference is referred to as binocular disparity and provides a strong depth perception cue in humans.

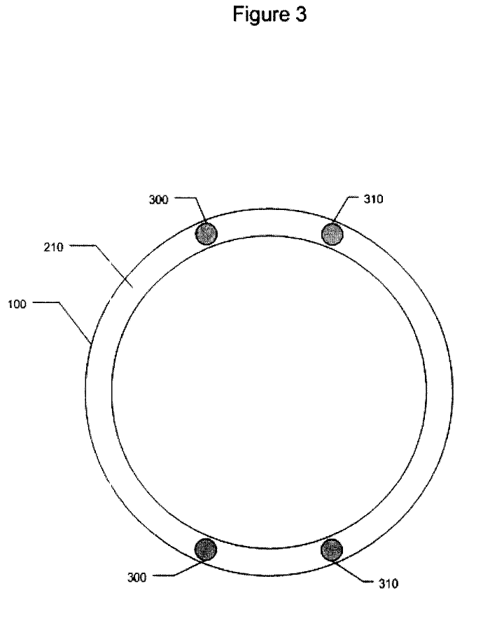

FIG. 3 shows the distal end of the cannula with an alternative arrangement of two cannula-based image capture sensors located at opposite ends of the geometric chord of the circumference of a circular cannula with chord length much less that the diameter of the cannula (i.e. this distance is know in the art as the interpupillary distance). In the figure, these sensors are shown as the right eye view 300 and left eye view 310. Controlling the distance between the image sensors and their position relative to the surgical instrument inserted through the cannula allows overlap in these sensors field of view. This in turn facilitates the use of cannula-based image sensors so located as image sources for a surgical stereo viewer. Illumination can be provided via a ring light 210 within the cannula 100 as well as the other embodiments mentioned. A second set of stereo sensors is also shown for the same purposes as the monocular view.

This image sensor geometry will enable surgeons to perceive the strong depth cues created by binocular disparity when viewing the output from these sensors using a compatible stereo image viewer. As is known in the art, the binocular disparity can be determined by the ratio of the interpupillary distance to the working distance. It is therefore possible to design cannulas under the present invention with various image capture sensor geometries depending on the expected imaging system usage (e.g. maximal field of view or stereo vision).

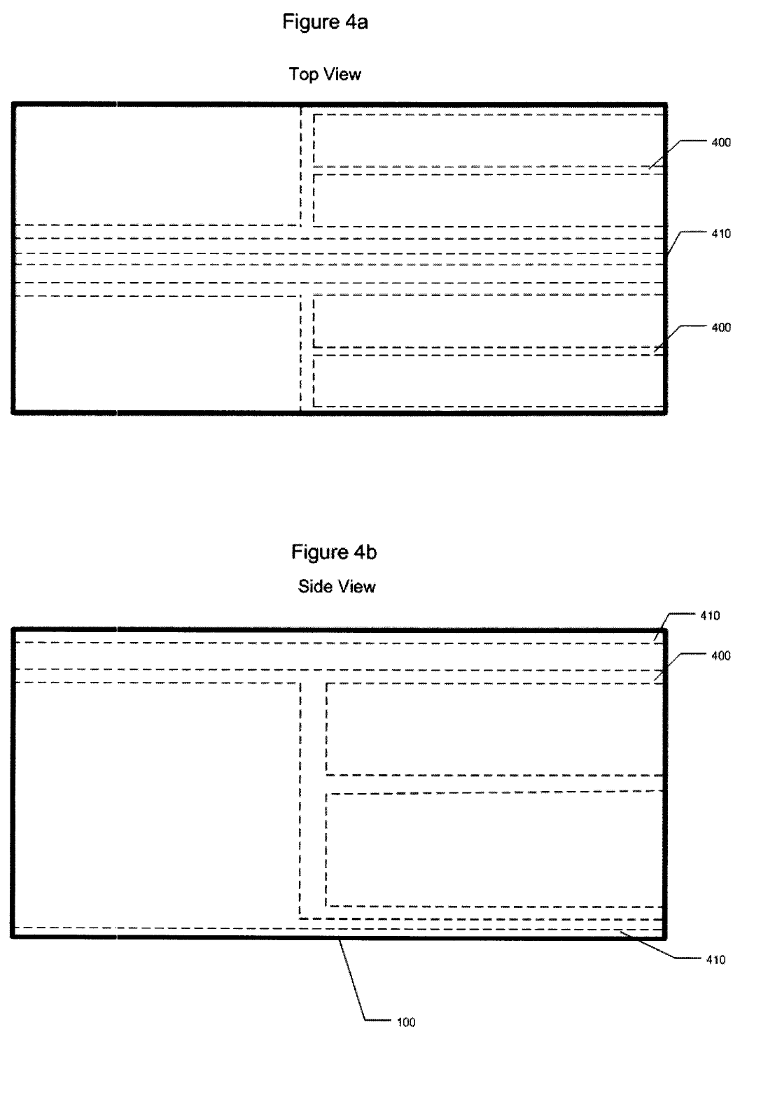

FIG. 4a shows a top view and FIG. 4b a side view of the light channels 400 to allow for a single input to provide illumination to multiple light outputs in the embodiment where the light source is external to the cannula 100. The camera connection conduit 410 is also shown.

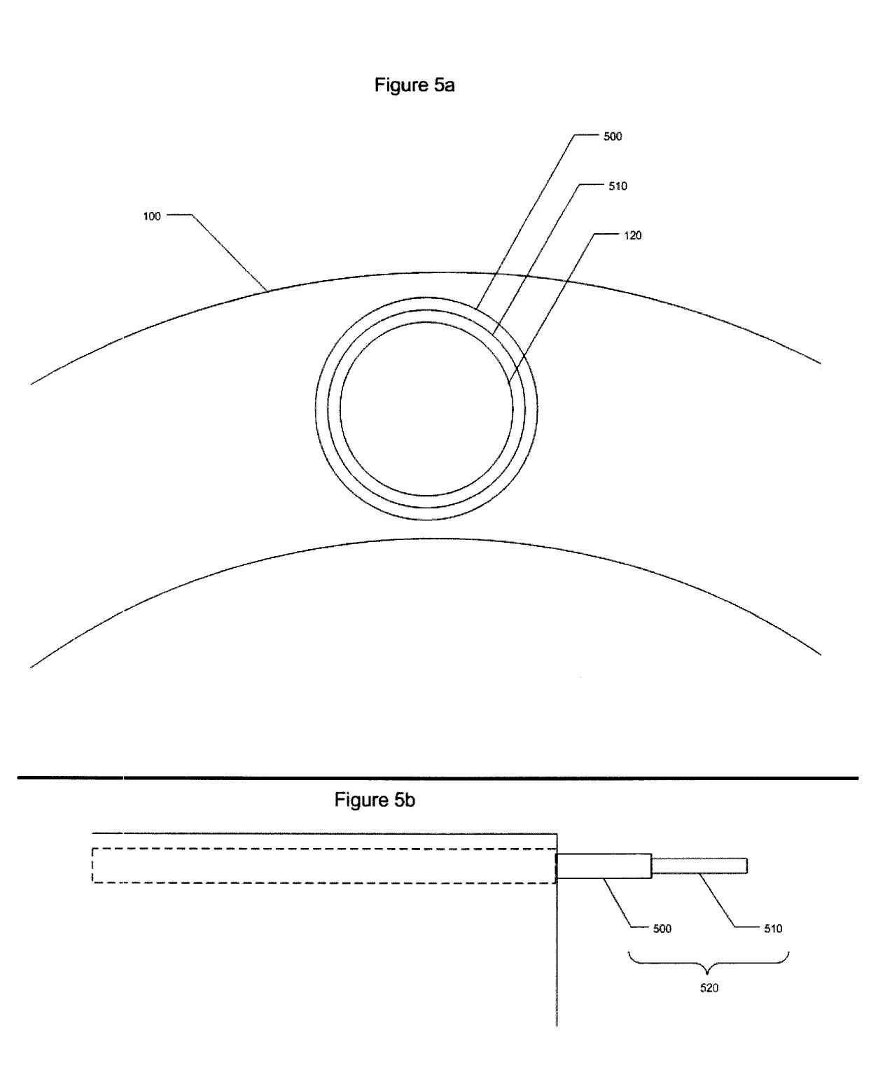

Optics and/or mechanics can be used to focus, magnify and/or orient the image capture sensor's field of view depending on the desired optical system properties. FIG. 5a shows an example of an image capture sensor with a telescoping ring in cross-section and in FIG. 5b as a telescoping sleeve in profile view. In both views, an inner telescoping ring 510 and an outer telescoping ring 500 allow the camera 120 to move closer to the target area, provide for focusing, or allow proper distancing for stereo viewing. Both of these components, taken together, comprise an optical probe 520 telescoping camera. These and other optical/mechanical variations of cannula-based illumination and/or image capture sensor(s) are covered under the present invention.

The present invention's use of image sensor(s) in the cannula has similar benefits to those previously described for cannula illumination sources.

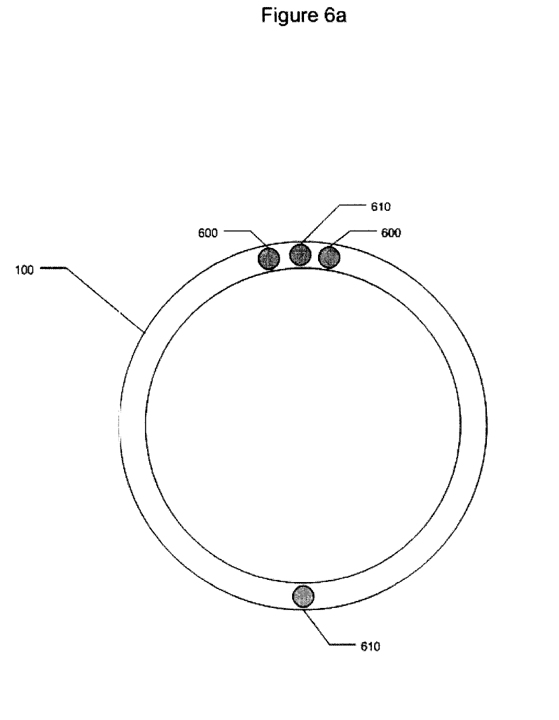

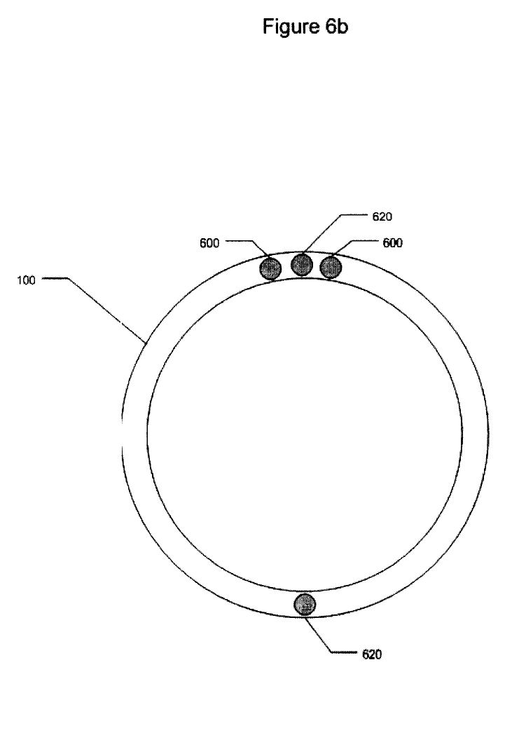

FIG. 6 shows the proximal end of the cannula 100 that is outside the patient's body (i.e. the cannula's proximal end). This is the surgical instrument port through which surgical instruments are inserted into the patient's body cavity. The gas port used to connect the cannula to the gas source and the with gas flow control valve used to control gas flow are also observed. These are traditional functions of surgical cannulas known in the art.

FIG. 6a also shows the addition of an external illumination source connector 600 to which an external illumination source may be connected. Illumination source connectors such as these are commonly used on surgical laparoscopes for this purpose as previously described. By providing similar illumination connectors 600, the present invention can be used with traditional surgical light sources (e.g. Xenon light sources) when visible illumination is required. It is also the intention of this invention to support the use of non-traditional illumination sources through this surgical cannula that can be used in conjunction with the traditional visible illumination provided by laparoscopes known in the art. An electronic connection port 610 for the camera is also shown. Multiple connectors are shown to provide an optional view of the target area. Theses connectors are well known in the art (see reference above on 1 mm cameras).

FIG. 6b shows the use of an optical relay 620 to enable the image to be relayed onto the image sensor in an external camera connected to the proximal end of the cannula.

Another embodiment is an image capture sensor configuration where the sensor is mounted on the proximal end of the cannula 100 and remains outside the body during operation. The image is relayed from the distal end of the cannula image sensor to the proximal end using well know technologies currently in use in ridged and flexible laparoscopes and endoscopes such as discrete optics and coherent fiber optic bundles. As image capture sensors continue to be miniaturized, consume less power and reduced in cost, image capture sensors can be located at the distal end of the cannula as an alternative embodiment. Moving the sensor to the distal end of the cannula is analogous to what has occurred with traditional laparoscope sensors. Instead of mounting the image sensors external to the body on the proximal end of the laparoscope, the Olympus Endo-Eye Laparoscope mounts the image sensor on the distal end of the laparoscope where it operates inside the body. The image sensor could be built into the cannula distal end or located in a camera externally connected to the cannula. The later approach would allow the cannula to be disposable (i.e. manufactured for less cost) and the camera with image sensor to be reusable for other procedures.

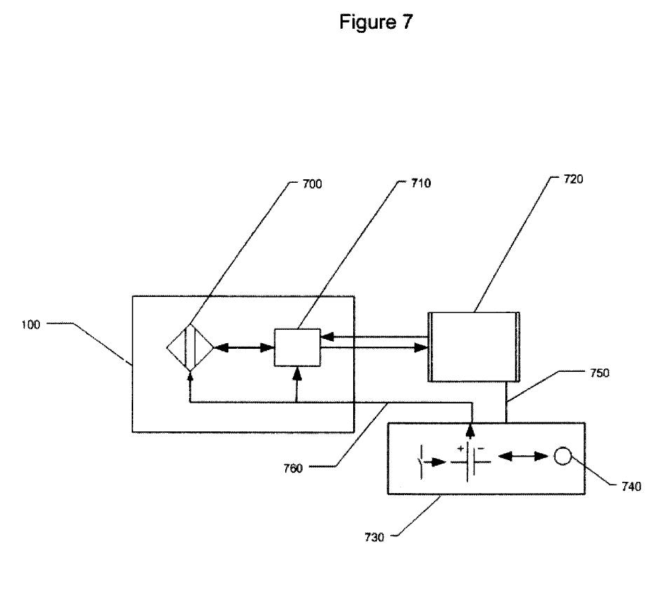

FIG. 7 shows a system that allows for determining the position of the different cameras between different cannulas to allow proper orientation for stereo viewing. There are products on the market that sense its position and posture and can be made to communicate this information.

One of these devices, for example, is the FalconGX 6DOF Sensor Module, a product of O-Navi LLC, Micro Avionics Group, San Diego, Calif. This device exhibits the behavior described with respect to FIG. 7, reporting angular rate and acceleration along mutually orthogonal x, y, and z axes, with increased output corresponding to movement in the directions. Sensor module 700 may also detect other conditions, such as proper operating temperature (indicating warm-up is completed and equipment accuracy can be assumed). Refer to the commonly-assigned copending U.S. Provisional Patent Application Ser. No. 60/863,976, filed Nov. 2, 2006. This sensor assembly is located within the wall of the cannula 100 and the position of the sensor is known (via its assembly), relative to the position of the cameras.

Because it detects motion rate and acceleration, rather than merely tilt, sensor module 700 runs continuously, receiving power from an external power supply 730, typically some type of battery or other storage cell. Alternately, AC power could be provided externally and converted to the needed DC levels; however, portable power has significant advantages for handling and ease of use. A power indicator 740 is used to determine the on/off state. Power can be connected directly to the sensor 760 or, alternatively, to the external processor 720 via standard power connections 750. A control logic processor is contained within the external system 720 is in communication with sensor module 700, using communication means 710, for obtaining the angular rate and acceleration data at regular sampling intervals. With the FalconGX module, for example, sampling can be performed at 50 times per second. Control logic processor contained within the external system may also be an on-board microprocessor or other dedicated control logic device. Storage can be provided for within the external system 720. A display is provided with these external systems and can be used as an indicator responsive to orientation data from sensor module 700 and providing some visible and/or audible indication of relative position, as an aid to help the technician to ascertain in which direction adjustment is needed. An operator interface is shown in commonly-assigned copending U.S. Provisional Patent Application Ser. No. 60/863,976, filed Nov. 2, 2006.

This system can now provide real-time alignment of the cameras from 2 different cannulas and now provide stereo viewing. Fine tuning of the cameras can be obtained by several means. One means is by using the telescopic capability of the cameras shown in FIG. 5. Another is by manual rotation of the cannulas when they are inserted initially. The display on the external system can indicate the proper positioning and be used for moving the cannula/camera system into proper orientations as shown in commonly-assigned copending U.S. Provisional Patent Application Ser. No. 60/863,976, filed Nov. 2, 2006.

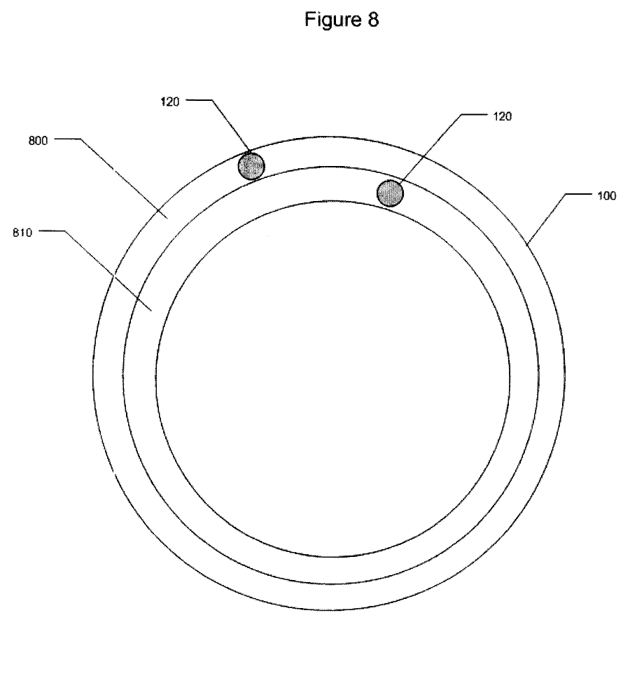

FIG. 8 shows an embodiment where a sleeve within the cannula 100 can be rotated for positioning of cameras. The outer sleeve of the cannula 800 is fixed while the inner sleeve 810 can be rotated to allow for customized positions that can optimize viewing or positioned for stereo visualization.

The invention has been described in detail with particular reference to certain preferred embodiments thereof, but it will be understood that variations and modifications can be effected within the scope of the invention.

### PARTS LIST

100 cannula for laparoscopic surgery
110 light within the cannula
120 camera channel within cannula
130 camera within cannula
140 image capture sensor
210 ring light
300 right-eye stereo camera view
310 left-eye stereo camera view
400 light channel
410 camera conduit
500 outer telescoping ring of camera
510 inner telescoping ring of camera
520 optical probe
600 external illumination source connector
610 electronic connection port
620 optical relay
700 sensor module
710 communications means
720 external system (including display)
730 external power supply
740 power indicator
750 power connection for wired solution
760 sensor
800 outer sleeve of cannula
810 rotating inner sleeve of cannula

1. A medical cannula comprising:
a first sleeve;
a center punch which fits within said sleeve for providing an opening in a body for said sleeve and said center punch; and
at the least one camera channel in said sleeve for viewing inside said body.

2. The medical cannula as in claim 1 further comprising:
at least one light channel in said sleeve.

3. The medical cannula as in claim 1 further comprising:
a plurality of camera channels in said sleeve.

4. The medical cannula as in claim 3 further comprising:
wherein at least two of said camera channels are located 180° apart on said sleeve.

5. The medical cannula as in claim 3 further comprising:
wherein at least two of said camera channels provide stereoscopic imaging.

6. The medical cannula as in claim 1 wherein an optical relay is in said camera channel.

7. The medical cannula as in claim 1 comprising:
a second sleeve with at least one camera channel in said second sleeve, rotatable about said first sleeve.

8. The medical cannula as in claim 1 further comprising:
a second sleeve concentric to said first sleeve;
at the least one camera channel in and said second sleeve for viewing inside said body; and
wherein said second sleeve it is rotatable about said first sleeve for the purpose of changing an optical orientation of a camera in said camera channel, in said second sleeve, relative to a camera in said camera channel, in said first sleeve.

9. The medical cannula as in claim 8 further comprising:
a third sleeve concentric to said first and said second sleeve.

10. The medical cannula as in claim 1 further comprising:
at least one camera or sensor in said at least one camera channel.

11. The medical cannula as in claim 1 further comprising:
at least one optical probe in said at least one camera channel connected to a camera.

12. The medical cannula as in claim 1 further comprising:
at least one of the light source in said sleeve.

13. The medical cannula as in claim 12 further comprising:
wherein said light source is comprised of a light pipe connect to an external light source.

14. The medical cannula as in claim 1 further comprising:
a second medical cannula comprising:
a first sleeve;
a center punch which fits within said sleeve for providing an opening in a body for said sleeve and said center punch; and
at the least one camera channel in and said sleeve for viewing inside said body.

15. The medical cannula as in claim 14 further comprising:
wherein said the first cannula provides a first image and said second cannula provides a second image.

16. The medical cannula as in claim 15 further comprising:
wherein said first and second image provide a stereoscopic view.

17. The medical cannula as in claim 16 further comprising:
wherein an xyz sensor stabilizes said stereoscopic view.
